(19) United States
(12) Patent Application Publication
(10) Pub. No.: US 2025/0152733 A1
(43) Pub. Date: May 15, 2025

(54) ANTIBIOTIC AMPHIPHILIC NANOPARTICLE AND METHODS OF USING THE SAME AGAINST GRAM-NEGATIVE AND/OR GRAM-POSITIVE BACTERIA

(71) Applicant: Indiana University Research and Technology Corporation, Bloomington, IN (US)

(72) Inventors: Yan Yu, Bloomington, IN (US); Jared T. Wiemann, Bloomington, IN (US); Swagata Bhattacharyya, Bloomington, IN (US)

(21) Appl. No.: 18/837,694

(22) PCT Filed: Feb. 8, 2023
(86) PCT No.: PCT/US2023/062203

§ 371 Date: Aug. 12, 2024

Related U.S. Application Data
(60) Provisional application No. 63/307,890, filed on Feb. 8, 2022.

(51) Int. Cl. A61K 47/69; A61P 31/04

(52) U.S. Cl. CPC A61K 47/6929; A61P 31/04

(57) ABSTRACT

“Two-faced” amphiphilic Janus nanoparticles that have different surface chemistries on two hemispheres. One hemisphere of the Janus nanoparticles is functionalized with a hydrophobic moiety. The other hemisphere of the nanoparticles is functionalized with either a cationic antibiotic or cationic polymer. Janus nanoparticles effectively inhibit the growth of both Gram-negative and Gram-positive bacteria at picomolar concentrations and may be used as a broad-spectrum antibiotic on surfaces or to treat bacterial infections in patients.

### STATEMENT OF GOVERNMENTAL RIGHTS

This invention was made with government support under 1705384 awarded by the National Science Foundation. The government has certain rights in the invention.

### FIELD OF THE INVENTION

The present disclosure relates nanoparticles, particularly in the field of antimicrobials and antimicrobial treatments.

### BACKGROUND

According to CDC, as of 2019, there were an average of 2.8 million cases of antibiotic-resistant infections every year leading to approximately 35,000 deaths per year in the United States alone, establishing antibiotic-resistant bacteria as a major risk to public health.¹ Over time, strains of bacteria have arisen that have become resistant to these antibiotics, while other bacteria, especially Gram-negative bacteria, have intrinsic resistance to large swaths of antibiotics.^{2, 3} Accordingly, there exists a clear need for new antibiotics.

To address the problem of antibiotic resistant bacteria some researchers have developed nanomaterials to treat bacteria-related disease. A major advantage of antibiotic nanomaterials compared to traditional small-molecule antibiotics is that many nanomaterials are thought to kill bacteria through mechanisms that are less likely to induce bacterial resistance. Those mechanisms include physical binding to DNA⁴⁻⁶, inducing production of reactive oxygen species (ROS)^{7, 8}, or direct penetration of the bacterial membranes⁹⁻¹¹. Studies have shown that the antibiotic potency of nanoparticles is largely determined by their surface chemistry.^{12, 13} In general, particles displaying cationic charges are more disruptive to bacterial membranes and hence more potent in inhibiting bacterial growth than anionic particles, possibly due to the stronger electrostatic attractions between cationic particles and the anionic bacteria cell wall or outer membrane.^{4, 14, 15}

Some previous studies have shown that amphiphilic nanoparticles, which display a uniform mixture of cationic and hydrophobic ligands on the surface, have high selectivity for bacteria cells over mammalian cells and are effective against several Gram-negative bacteria and Gram-positive bacteria.¹⁶⁻¹⁸ For example, Gupta, et al. showed that 35 nm amphiphilic polymeric nanoparticles can inhibit growth of P. aeruginosa at particle concentrations ranging from 64 to 128 nM. Some researchers have also attempted to tune the selectivity of gold nanoparticles for either Gram-negative or Gram-positive bacteria by varying the ratio between anionic and cationic ligands on the particle surface.¹⁹ In a separate study, researchers studied nanoparticle-induced inhibition of bacterial growth by varying the amphiphilicity of particles using different alkyl chain lengths on amphiphilic ligands.¹⁸ In both strategies, the nanoparticles were made to display a uniform mixture of charged and hydrophobic ligands.

While progress has been made in the development of antibacterial nanoparticles, further improvements are still needed. Aspects of the invention disclosed herein address this need.

### SUMMARY OF THE INVENTION

A first aspect of the invention includes antibacterial Janus nanoparticles have a charged hemisphere and a hydrophobic hemisphere.

A second aspect of the invention includes methods for killing Gram-positive and Gram-negative bacterial using antibacterial Janus nanoparticles.

A third aspect of the invention includes methods of treating bacterial infections using antibacterial Janus nanoparticles.

A first embodiment is a Janus nanoparticle having a charged moiety and a hydrophobic moiety, where the charged moiety and the hydrophobic moiety are attached to the nanoparticle independently of one another.

A second embodiment is a Janus nanoparticle where the charged moiety is attached to a first hemisphere and the hydrophobic moiety is attached to the opposite hemisphere of the nanoparticle.

A third embodiment is a Janus nanoparticle where the hydrophobic moiety includes at least one hydrophobic alkyl chain.

A fourth embodiment is a Janus nanoparticle where the hydrophobic alky chain of the hydrophobic moiety is a linear chain, branched chain, or polymer.

A fifth embodiment is a Janus nanoparticle where the hydrophobic alkyl moiety includes 4 or more carbon-carbon bonds.

A sixth embodiment is a Janus nanoparticle where the charged moiety is an antibiotic.

A seventh embodiment is a Janus nanoparticle where the antibiotic is a polymyxin antibiotics, antimicrobial peptides, and steroid antibiotic, wherein the antibiotic exhibit a positive charge.

An eighth embodiment is a Janus nanoparticle where charged moiety is the antibiotic colistin.

A ninth embodiment is a Janus nanoparticle where the charged moiety is a cationic polymer.

A tenth embodiment is a Janus nanoparticle where the charged moiety is a cationic that is cationic poly(amidoamine), cationic linear polymers, cationic branched polymers, cationic dendrimers, cationic polypeptide, or other cationic nanoparticles.

An eleventh embodiment is a Janus nanoparticle capable of killing Gram-negative bacteria.

A twelfth embodiment is a Janus nanoparticle capable of killing Gram-positive bacteria.

A thirteenth embodiment is a method of killing bacteria, where the Janus nanoparticle is placed in contact with a bacteria cell.

A fourteenth embodiment is a method of killing bacteria where the Janus nanoparticle is placed in contact with a Gram-negative or Gram-positive bacteria.

A fifteenth embodiment is a method of treating a patient by providing the Janus nanoparticle to the patient diagnosed with a bacterial infection by administering at least one therapeutically effective dose of the least one compound to a patent, where the patient is diagnosed with a bacterial infection.

A sixteenth embodiment is a method of treating a patient diagnosed with a bacterial infection where the therapeutically effective dose of Janus nanoparticle administered is within the picomolar to micron-molar range.

A seventeenth embodiment is a method for treating a patient diagnosed with a bacterial infection where the bacterial infection is caused by a Gram-negative or a Gram-positive bacteria.

An eighteenth embodiment is a method for treating a patient diagnosed with a bacterial infection where the patient is a human or an animal.

A nineteenth embodiment is a method for treating a patient diagnosed with a bacterial infection where the therapeutically effect dose of Janus nanoparticles is administered topically, orally, or intravenously.

### BRIEF DESCRIPTION OF THE FIGURES

The accompanying drawings are included to provide a further understanding of the disclosure and are incorporated in and constitute a part of this specification, illustrate embodiments, and together with the description serve to explain the principles of the disclosure.

FIG. 1A. Schematic of PAMAM dendrimer/hydrophobic (dend/pho JP) and colistin/hydrophobic (col/pho JP) Janus nanoparticle fabrication method and illustration of control colistin uniform nanoparticles (col UNP), colistin/hydrophobic uniform nanoparticles (colpho UNP), and PAMAM dendrimer uniform nanoparticles (dend UNP).

FIG. 1B. Scanning electron microscopy image showing Janus geometry of gold cap after thermal evaporation.

FIG. 1C. Hydrodynamic diameter of colistin conjugated nanoparticles in 2 mM HEPES (pH 7.2). Scale bar: 100 nm

FIG. 1D. Zeta potential of colistin conjugated nanoparticles in 2 mM HEPES (pH 7.2). Scale bar: 100 nm.

FIG. 1E. Hydrodynamic diameter of PAMAM dendrimer conjugated nanoparticles in 2 mM HEPES (pH 7.2). Scale bar: 100 nm.

FIG. 1F. Zeta potential of PAMAM dendrimer conjugated nanoparticles in 2 mM HEPES (pH 7.2). Scale bar: 100 nm.

FIG. 2A. Viability of Escherichia coli in the presence of colistin/hydrophobic Janus nanoparticles (col/pho JP), colistin/hydrophobic uniform nanoparticles (colpho UNP), and colistin uniform nanoparticles (col UNP) as determined by growth-based viability assay.

FIG. 2B. Viability of Vibrio cholerae in the presence of colistin/hydrophobic Janus nanoparticles (col/pho JP), colistin/hydrophobic uniform nanoparticles (colpho UNP), and colistin uniform nanoparticles (col UNP) as determined by growth-based viability assay

FIG. 2C. Viability of Staphylococcus aureus in the presence of colistin/hydrophobic Janus nanoparticles (col/pho JP), colistin/hydrophobic uniform nanoparticles (colpho UNP), and colistin uniform nanoparticles (col UNP) as determined by growth-based viability assay.

FIG. 2D. EC₅₀ for each nanoparticle and bacteria based on data from FIGs. A-D.

FIG. 3A. Viability of E. coli in the presence of PAMAM dendrimer/hydrophobic Janus nanoparticles (dend/pho JP) and PAMAM dendrimer uniform nanoparticles (dend UNP) as determined by the growth-based viability assay.

FIG. 3B. Viability of B. subtilis in the presence of PAMAM dendrimer/hydrophobic Janus nanoparticles (dend/pho JP) and PAMAM dendrimer uniform nanoparticles (dend UNP) as determined by the growth-based viability assay.

FIG. 3C. EC50 for each dendrimer nanoparticle and bacteria based on data from FIGS. 3A. and 3B.

FIG. 4A. Merged images (right panel) of propidium iodide staining (center panel) of E. coli (DIC, left panel) after incubation with 64 pM colistin/hydrophobic Janus nanoparticles (col/pho JPs). Scale bar: 10 μm.

FIG. 4B. Merged images (right panel) of propidium iodide staining (center panel) of E. coli (DIC, left panel) after incubation with buffer only, no nanoparticles present. Scale bar: 10 μm.

FIG. 4C. Merged images of DIC and propidium iodide fluorescence images of E. coli after incubation with listed concentrations of colistin/hydrophobic uniform nanoparticles (colpho UNPs). Scale bar: 10 μm.

FIG. 4D. Merged images of DIC and propidium iodide fluorescence images of E. coli after incubation with listed concentrations of colistin uniform nanoparticles (col UNPs). Scale bar: 10 μm.

FIG. 5A. Merged images (right panel) of propidium iodide staining (center panel) of B. subtilis (DIC, left panel) after incubation with 64 pM colistin/hydrophobic Janus nanoparticles (col/pho JP). Scale bar: 10 μm.

FIG. 5B. Merged images (right panel) of propidium iodide staining (center panel) of B. subtilis (DIC, left panel) after incubation with buffer only, no nanoparticles present. Scale bar: 10 μm.

FIG. 6A-B. Scanning electron microscopy (SEM) images of E. coli without nanoparticles. Scale bar: 0.5 μm.

FIG. 6C-E. Scanning electron microscopy (SEM) images of E. coli after interaction with 64 pM col/pho Janus nanoparticles. Arrows indicate deformation of the bacterial cell envelop. Scale bar: 0.5 μm.

FIG. 6F. Scanning electron microscopy (SEM) images of E. coli after interaction with 128 pM Janus nanoparticles. Arrow indicates ruptured bacterial cell envelop. Scale bar: 0.5 μm.

FIG. 7A-B. Scanning electron microscopy (SEM) images of B. subtilis without nanoparticles. Scale bar: 0.5 μm.

FIG. 7C-E. Scanning electron microscopy (SEM) images of B. subtilis after interaction with 64 pM col/pho Janus nanoparticles. Arrows indicate deformation of the bacterial cell envelop. Scale bar: 0.5 μm.

FIG. 7F. Scanning electron microscopy (SEM) images of B. subtilis after interaction with 128 pM Janus nanoparticles. Arrow indicates ruptured bacterial cell envelop. Scale bar: 0.5 μm.

FIG. 8. Graph depicting difference in florescence intensity of Reactive Oxygen Species (ROS) from colistin hydrophobic Janus Particles and colistin hydrophobic Uniform Particles. Fluorescence intensity from individual bacteria was measured as a function of concentration.

### DETAILED DESCRIPTION

We identified a major drawback with the prior strategies in developing antibacterial nanoparticles. Specifically, because the prior strategies include a uniform mixture of charged and hydrophobic ligands across the surface of the nanoparticle, the hydrophobic ligands next to charged ligands hinder the electrostatic attraction of the nanoparticles to bacteria and, meanwhile, the charged ligands can prevent the hydrophobic force-driven nanoparticle disruption of the bacterial membranes. As disclosed herein, we successfully circumvented this challenge by designing and creating Janus nanoparticles which spatially separate the charged ligands and hydrophobic ligands on the surface of single nanoparticles. Janus nanoparticles, named after the Roman god Janus with two faces in opposite directions, are anisotropic nanoparticles with two distinct hemispheres.²⁰ A major advantage of the Janus nanoparticles compared to conventional nanoparticles is that we are able to spatially separate the hydrophobic and charged ligands onto two separate hemispheres on the nanoparticles. This allows the nanoparticles to be attracted to lipid bilayers through electrostatic interaction without interference from the hydrophobic ligands, and then reorient to insert into bio-membranes from their hydrophobic hemisphere without interference from the charged hemisphere.²¹

As described herein we developed two exemplary types of amphiphilic Janus nanoparticles. The Janus nanoparticles are hydrophobic on one hemisphere in both types. In the exemplary Janus particles described below the hydrophobic hemisphere includes an 18-carbon alkyl chain with a thiol (SH) end group. Other hydrophobic ligands for use in the antibacterial Janus particles include, but are not limited to, hydrophobic alkyl chains. Those of skill in the art will appreciate that the hydrophobic alkyl chains may include linear chains, branched chains, or polymers. Preferred hydrophobic alkyl moieties for use in the Janus nanoparticles include 4 or more carbon-carbon bonds with alkyl moieties having a backbone of greater than 4 carbon-carbon bonds in length most preferred.

The cationic hemisphere of the two exemplary Janus nanoparticles was conjugated with either colistin or poly(amidoamine) (PAMAM) dendrimer. Colistin is a cationic antibiotic molecule that is often use as a last resort to kill Gram-negative bacteria. (Available from Sigma Aldrich). Colistin is thought to work by binding lipopolysaccharide in the cell envelope.^{24, 25} Poly(amidoamine) (PAMAM) dendrimer is a molecule with similar charge and molecular weight as colistin but has not a clinically approved for use as an antibiotic. (Available from Sigma Aldrich). Other charged moieties may be conjugated with the Janus nanoparticle. Specifically, the charged moiety may include, but are not limited to, an antibiotic such as cationic lipopeptides (such as polymyxin B and polymyxin E (also known as colistin), antimicrobial peptides (such as ambicin, also known as nisin), Gramicidins (such as Gramicidin S), Ceragenins, Peptidomimetic-based antimicrobial cationic amphiphiles, Benzophenone-based antimicrobial cationic amphiphiles, Xanthone-based antimicrobial cationic amphiphiles, Aminoglycoside-derived antimicrobial cationic amphiphiles, or steroid antibiotics. One of skill in the art will appreciate that a variety of antibiotic molecules may be used in the disclosed Janus particles where the antibiotic exhibits a positive charge. The disclosed Janus nanoparticles may also include a cationic polymer as the charged moiety to be placed opposite the hydrophobic moiety. Cationic polymers may include cationic poly(amidoamine), cationic linear polymers, cationic branched polymers, cationic dendrimers, cationic polypeptide, or other cationic nanoparticles.

Core particles for use in creating the antibacterial Janus nanoparticles of the invention include, but are not limited to, inorganic particles, polymeric particles, and metal particles. One of ordinary skill in the art would appreciate that a variety of materials capable of use as microparticles and/or nanoparticles may also be utilized as carrier particles in the disclosed invention. In one embodiment of the invention, the carrier particle is silica or polystyrene.

The disclosed Janus particles have shown to be effective in killing both Gram-negative and Gram-positive bacteria. Examples of Gram-negative bacteria susceptible to the disclosed Janus particles include, but are not limited to, strains of Escherichia coli and Vibrio cholerae. Examples of Gram-positive bacteria susceptible to the disclosed Janus particles include, but are not limited to, strains of Bacillus subtilis and Staphylococcus aureus.

The disclosed antimicrobial properties of the disclosed Janus particles may utilized in killing bacteria on exposed surfaces as well as treating bacterial infections in a human or animal. The concentration of antimicrobial Janus particles needed to reduce the population of bacteria present (the EC50) is significantly less than that observed by prior art nanoparticles. For both Gram-negative and Gram-positive bacteria, the disclosed Janus particles were much more potent than conventional antibiotic nanoparticles with uniform surface chemistry. Picomolar and nanomolar quantities of antimicrobial Janus particles showed significant reduction in bacterial populations.

When used to treat a bacterial infection in a human or animal, antimicrobial Janus particles may be administered in a therapeutically effective amount via a route of administration most applicable to the site of the infection. One skilled in the art would appreciate that the Janus nanoparticles may be administered to the patient diagnosed with a bacterial infection topically, orally or intravenously, as well other approved administrative routes.

The dendrimer/hydrophobic nanoparticles also described herein demonstrate that the potency of Janus nanoparticles is applicable to cationic ligands in general and not limited to cationic antibiotic molecules. Janus nanoparticles were found to be more potent antimicrobials for both Gram-negative and Gram-positive bacteria than nanoparticles with uniform surface chemistry. The EC₅₀ of Janus nanoparticles is in the picomolar concentration range and 3-18 times lower than EC₅₀ of conventional uniform nanoparticles.

### Abbreviations and Definitions

Abbreviations

| Abbreviation | Definition |
|---|---|
| PAMAM | Poly(amidoamine) dendrimer |
| col/pho JP | Colistin/hydrophobic Janus nanoparticle |
| colpho UNP | Colistin/hydrophobic uniform nanoparticle |
| col UNP | Colistin uniform nanoparticle |
| dend/pho JP | Poly(amidoamine) dendrimer/hydrophobic Janus |
| | nanoparticle |
| dend UNP | Poly(amidoamine) dendrimer uniform nanoparticle |
| EC50 | Half-maximum effective concentration |
| ROS | Reactive oxygen species |
| E. coli | Escherichia coli |
| V. cholerae | Vibrio cholerae |
| B. subtilis | Bacillus subtilis |
| S. aureus | Staphylococcus aureus |
| DIC | Differential interference contrast |
| pM | Picomolar (10⁻¹² moles/liter) |

Unless states otherwise of clearly implied otherwise the term ‘therapeutic amount’ is the amount is an amount of the compound that either in a single dose or as part of course of treatment has a therapeutic effect on a patient.

Unless states otherwise of clearly implied otherwise the term ‘patient’ refers to either a human or an animal

### EXAMPLES

### Example 1: Nanoparticle Fabrication

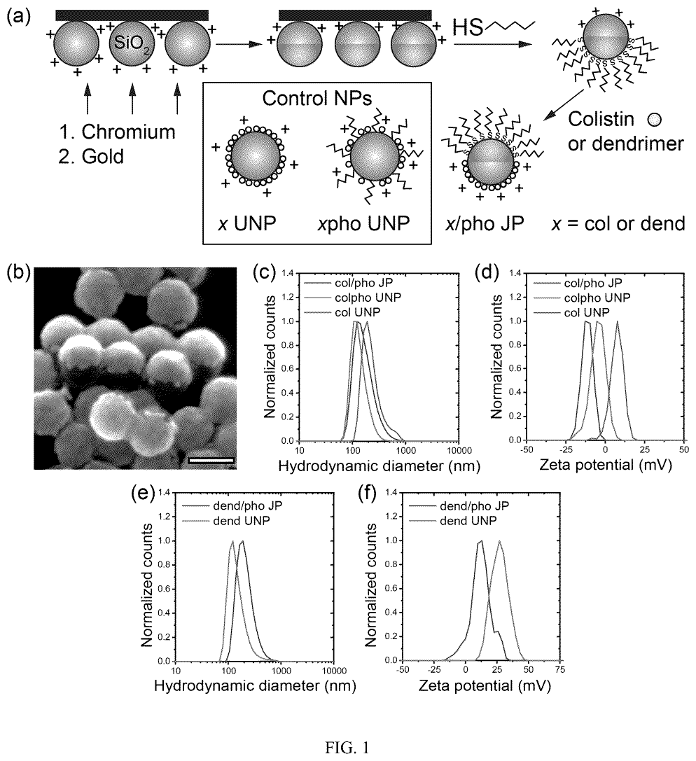

Amphiphilic cationic Janus nanoparticles were fabricated using the techniques as previously described. (Lee, et al., ACS nano 2018, 12(4), 3646-3657). Cationic silica nanoparticles (100 nm in diameter) were drop cast onto piranha etched microscope slides to make a sub-monolayer of particles. An Edwards thermal evaporation system (Nanoscale Characterization Facility at Indiana University) was used to sequentially deposit thin layers of chromium (5 nm) and gold (25 nm) onto one hemisphere of the nanoparticles. FIG. 1A, left panel. Particle monolayers were immediately immersed in 2 mM 1-octadecanethiol for at least 12 h before use to make the gold caps on particles hydrophobic. FIG. 1A, middle panel. Particles were sonicated off the microscope slides and subjected to differential centrifugation (4 times at 100×g, 4 times at 500×g) to remove metal bridging aggregates formed during thermal evaporation. Janus particle gold coating was assessed by scanning electron microscopy (Nanoscale Characterization Facility at Indiana University). Amphiphilic silica nanoparticles were made prior to colistin conjugation. Briefly, octadecyltrimethoxysilane and 1M HCl were added dropwise to THF to prepare a solution containing 22 mM octadecyltrimethoxysilane and 0.6 vol % of HCl. Cationic silica nanoparticles were resuspended in 8:1 (v/v) hexanes:octadecyltrimethoxysilane solution with vigorous stirring for 1 h at room temperature. The resulted amphiphilic silica nanoparticles were washed 3 times with ethanol and 3 times with water before further surface modification.

To conjugate PAMAM dendrimer or colistin, nanoparticles (amphiphilic Janus nanoparticles, amphiphilic cationic silica particles, or cationic silica nanoparticles) were washed 3 times with 10 mM HEPES buffer (pH 7.4) and resuspended with 10% glutaraldehyde in 10 mM HEPES buffer for 90 min at room temperature with gentle rotation. After glutaraldehyde activation of amine groups on the particle surface, the particles were washed three times with 10 mM HEPES buffer to remove excess glutaraldehyde. Particles were then resuspended with colistin or PAMAM dendrimer (1 mg/mL in 10 mM HEPES buffer) for 60 min at room temperature with gentle rotation. Particles were washed three times again with 10 mM HEPES followed by three times washing with 2 mM HEPES 25 mM NaCl buffer to remove unreacted colistin or PAMAM dendrimer. Particles were stored at 4° C. until use. Hydrodynamic radius and zeta potential of all particles were characterized using a Malvern Zetasizer (Nanoscale Characterization Facility at Indiana University). Concentration of particles was measured using Particle Metrix ZetaView (Nanoscale Characterization Facility at Indiana University).

To try to understand how the spatial organization of surface ligands impacts nanoparticle antibiotic efficacy, we first fabricated amphiphilic Janus nanoparticles with colistin (referred to as col/pho JPs, FIG. 1A). Col/pho JPs were fabricated by coating one hemisphere of 100 nm aminated silica nanoparticles with chromium and then gold, which was subsequently conjugated with octadecanethiol to make the gold hemisphere more hydrophobic. FIG. 1B shows the Janus geometry of these particles after metal deposition using scanning electron microscopy. The gold hemisphere appears bright relative to the silica hemisphere signal due to gold's high electron density. Colistin was then conjugated to the nanoparticles using glutaraldehyde crosslinking of the primary amine groups on the silica hemisphere to L-diaminobutyric acid residues in colistin. As controls for spatial presentation of nanoparticle surface ligands, we also fabricated a uniform colistin nanoparticle and uniform amphiphilic colistin nanoparticle (referred to as col UNP and colpho UNP, respectively). Amphiphilic silica nanoparticles were first fabricated by conjugating octadecyltrimethoxysilane to the surface of the aminated silica nanoparticles. Then, colpho UNPs and col UNPs were made by conjugating colistin to amphiphilic silica nanoparticles and aminated silica nanoparticles, respectively, using glutaraldehyde crosslinking. The hydrodynamic diameter of the nanoparticles was measured using dynamic light scattering (FIG. 1C), each of which had narrow distributions of size indicating particles were monodisperse. Zeta potential measurements (FIG. 1D) were −10.5±0.9, −8±5, and +6.9±0.7 mV in 2 mM HEPES (pH 7.2) for col/pho JP, colpho UNP, and col UNP, respectively. Hydrophobic groups on surfaces have been known to decrease a material's zeta potential, explaining the more negative zeta potential of colpho UNPs compared to col UNPs.²⁶ We have previously reported that cationic, amphiphilic Janus nanoparticles without colistin conjugation have a negative zeta potential in water.²¹ This is likely due to (1) addition of hydrophobic molecules and (2) irregular electrophoretic mobility due to polarizability of the gold hemisphere.²⁷.

In addition to colistin-conjugated nanoparticles, we also fabricated dendrimer-coated nanoparticles. We selected the generation 1.0 PAMAM dendrimer in part because it has a similar charge and molecular weight as colistin. PAMAM dendrimer by itself is not a known antibiotic. Uniform PAMAM dendrimer nanoparticles (dend UNPs) and hydrophobic PAMAM dendrimer Janus nanoparticles (dend/pho JPs) were functionalized using glutaraldehyde conjugation as well. Hydrodynamic diameter measurements of the dendrimer nanoparticles revealed that the particles were well dispersed after functionalization (FIG. 1E). The zeta potentials were +10±2 mV and +25±2 mV for dend/pho JPs and dendUNPs, respectively (FIG. 1F). Compared to colistin nanoparticles, we observed an overall positive shift in zeta potential for the PAMAM dendrimer nanoparticles. This likely arises from two factors: (1) PAMAM dendrimer has a total of eight primary amines while colistin has only seven; (2) colistin contains a hydrophobic alkyl chain that could negatively shift the zeta potential.

### Example 2: Testing Antibacterial Properties of Colistin Conjugated Nanoparticles in Growth-Based Viability Assay

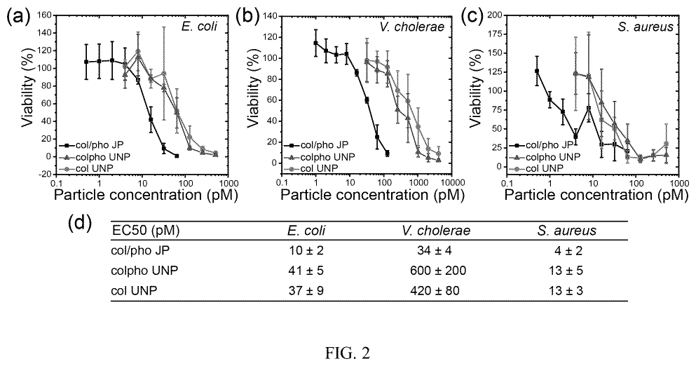

We compared the antibacterial properties of colistin nanoparticles against three lab strains of bacteria, namely Escherichia coli, Vibrio cholerae, and Staphylococcus aureus. To measure the viability of bacteria as a function of particle concentration, we used the growth-based viability assay as described by Qiu et al.²⁸ This assay measures viability by quantifying the delay of logarithmic bacterial growth as a result of fewer viable bacteria caused by pre-incubation with antibacterial nanoparticles. This particular assay was chosen as it has two benefits for comparing antibacterial properties of nanoparticles: (1) the initial exposure buffer is low ionic strength which minimizes aggregation effects between nanoparticles and (2) optical interference of nanoparticles is limited due to 10-fold dilution of exposure media into growth media. FIG. 2A-2C show the % viability of each bacteria as a function of nanoparticle concentration. All three of the colistin nanoparticles studied killed each of the bacteria beyond a concentration threshold. Notably, the nanoparticles were potent against both Gram-negative (E. coli and V. cholera) and Gram-positive bacteria (S. aureus), demonstrating the broad-spectrum capability of the colistin-conjugated nanoparticles. However, for all three species of bacteria tested, col/pho JPs reduced viability at lower concentrations than col UNPs or colpho UNPs.

To quantitatively compare the potency of nanoparticles, we fit dose-response plots with a Boltzmann-sigmoidal curve to calculate the EC50 (concentration where half of bacteria are killed, FIG. 2D). We found that col/pho JPs were approximately 4× (E. coli), 17× (V. cholerae), and 3× (S. aureus) more effective at killing bacteria than colpho UNPs. For each species tested, colpho UNPs and col UNPs had similar EC50s, which indicates that hydrophobicity alone is not enough to enhance the antibiotic properties of the nanoparticles. It is possible that close proximity of colistin and hydrophobic ligands attenuated the effect of particle hydrophobicity on bacteria viability. In contrast, this effect is not present for the case of col/pho JPs, as the two ligands are spatially segregated.

### Example 3: Testing Antibacterial Properties of PAMAM Dendrimer Conjugated Nanoparticles in Growth-Based Viability Assay

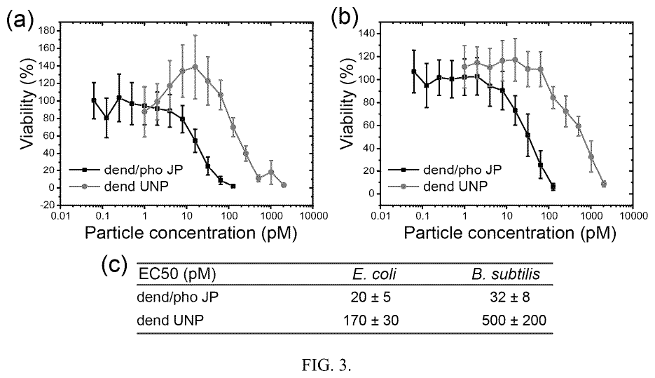

After observing that colistin-conjugated Janus nanoparticles were more potent than uniform nanoparticles, we wanted to see if this trend was consistent for particles conjugated with molecules that are not used as antibiotics. To accomplish this, we used particles coated with PAMAM dendrimer instead of colistin. While these molecules have been shown kill bacteria²⁹, they are not currently used as antibiotics in clinical applications. Upon performing viability assays with E. coli and B. subtilis, we observed that the Janus nanoparticles were able to kill bacteria at lower concentrations than the uniform nanoparticles (FIG. 3A-3B). EC50 calculated from dose-response curves in FIG. 3A-3B showed Janus nanoparticles were approximately 8× and 16× more potent than uniform nanoparticles for E. coli and B. subtilis, respectively (FIG. 3C). Together, these results support our hypothesis that amphiphilic Janus nanoparticles are generally more potent antibiotics than uniform nanoparticles.

### Example 4: Testing Cell Membrane Permeability of Colistin Conjugated Nanoparticles in Gram Negative Bacteria

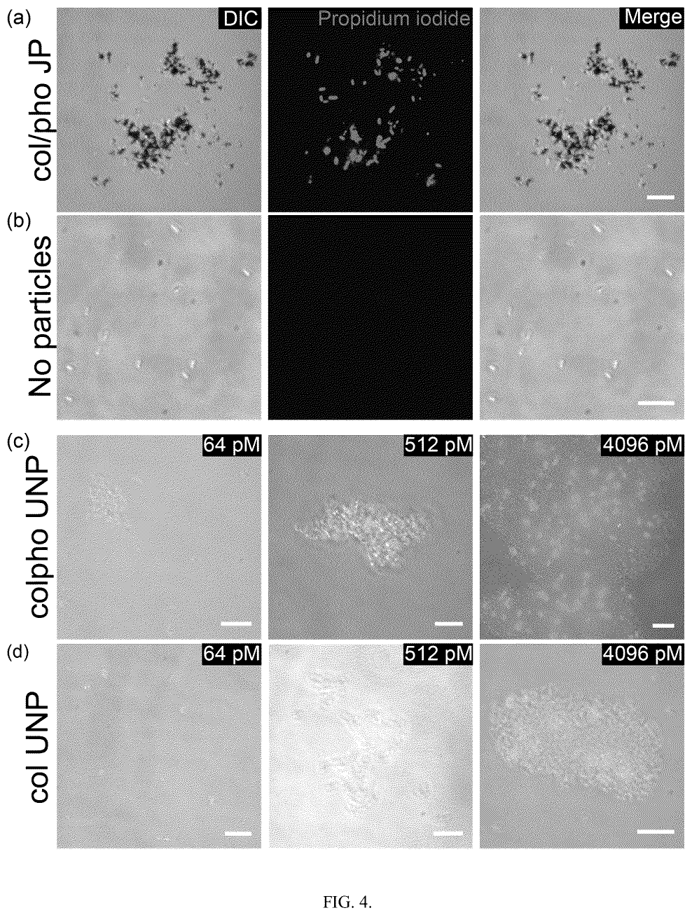

We sought to understand why our Janus nanoparticles were more effective antibiotics than uniform nanoparticles. Based on our previous work with synthetic lipid membranes^{21, 22}, we suspected that the nanoparticle's main antibiotic mechanism was disruption the bacteria cell envelope. We had previously tuned the interaction between amphiphilic Janus nanoparticles and lipid bilayers by adjusting the surface area ratio between the hydrophobic side and the charged side.²³ To see if particles permeabilized the outer membrane of bacteria, we used propidium iodide, a membrane-impermeable DNA stain that is weakly fluorescent in water. If the cell envelope has been permeabilized, propidium iodide diffuses into bacteria and intercalates with DNA, significantly increasing propidium iodide fluorescence intensity. After incubating col/pho JPs (64 pM) and bacteria (1×10⁸ cfu/mL) together for 2 hours at 37° C. with 280 rpm shaking, we then incubated each sample with 2 μM propidium iodide for 30 min. First, we used differential interference contrast microscopy (DIC) to image bacteria and the particles (FIG. 4A). col/pho JPs appear as dark spots due to their low optical transparency from the dense gold cap. DIC images revealed that col/pho JPs aggregated with E. coli to form large particle-E. coli complexes. This is in contrast to the case without particles where E. coli cells remained separated from one another (FIG. 4B). Fluorescence images revealed that a majority of E. coli bound to the col/pho JPs were stained with propidium iodide. This indicates that the col/pho JPs, at sufficient concentrations, are capable of penetrating the entire cell envelope of E. coli. Based on our viability results in the previous section, we expected that nearly all of the E. coli would be stained with propidium iodide in the presence of 64 pM col/pho JPs. There are two possible explanations for this result: (1) in the propidium iodide experiment, in order to obtain sufficient bacteria for imaging, ten times as many bacteria were used compared to the viability assays which leads to a smaller particle-to-cell ratio, and (2) for some cells, col/pho JPs may have only penetrated the outer membrane and thus the cell was not stained with propidium iodide. To address this, one could alternatively use vancomycin which has been fluorescently labeled with BODIPY to selectively label the cell wall. When the bacteria are intact, the outer membrane prevents access of vancomycin into E. coli and the cell wall remains unstained. However, if col/pho JPs disrupt the outer membrane of E. coli, one will see fluorescence from dye binding to the cell wall.

We then performed propidium iodide experiments with the uniformly coated nanoparticles, col UNPs and colpho UNPs (FIG. 4C-4D). Unlike the case with col/pho JPs, we did not observe significant aggregation of the particles and E. coli at 64 pM, though some (<20%) bacteria were stained with propidium iodide. However, when we increased the particle concentrations to 512 pM and 4096 pM, we saw large E. coli-nanoparticle aggregations, and E. coli was stained with propidium iodide in the presence of col UNPs and colpho UNPs. These results are consistent with what is observed in the viability assays which showed that Janus nanoparticles kill bacteria at lower concentrations than uniform nanoparticles. The propidium iodide data suggest that one explanation for the enhanced antibiotic potency of Janus nanoparticles is due to their ability to penetrate E. coli at lower bulk concentrations.

### Example 5: Testing Cell Membrane Permeability of Colistin Conjugated Nanoparticles in Gram Positive Bacteria

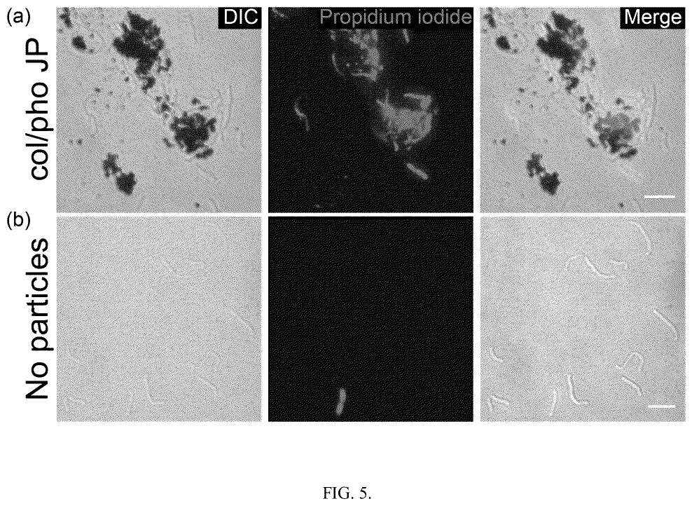

We investigated the cell envelope-penetrating properties of col/pho JPs against Gram-positive bacteria. For these experiments, we chose to use B. subtilis which is a common Gram-positive bacteria model. Similar to what we observed with E. coli, DIC images revealed col/pho JPs and B. subtilis clearly aggregated together (FIG. 5A) compared to B. subtilis alone, which remained well dispersed (FIG. 5B). Most B. subtilis cells in the aggregates were also stained with propidium iodide, indicating Janus nanoparticles are also capable of penetrating the Gram-positive cell envelope.

### Example 6: Antibacterial Effects of Amphiphilic Janus Nanoparticles in Disrupting the Cell Membrane Damage and Reactive Oxidative Stress (ROS) in Gram-Negative Bacteria

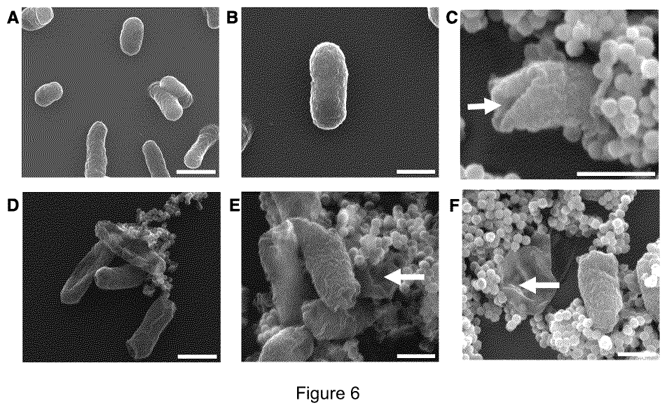

We treated the E. coli cells (1×10\hat{ }⁸) with and without col/pho Janus nanoparticles at varying concentrations. First, E. coli cells were fixed for scanning electron microscopy (SEM) measurements. SEM sample preparation was done by fixing the cells in Karnovsky's fixative, followed by 0.1M Phosphate Buffer for one hour, post fixation by osmium tetra oxide. The sample was washed through deionized water and a series of ethanol. E. coli bacteria without nanoparticle treatment did not have membrane damage. (FIG. 6A-6B)

Next, 64 pM of col/pho Janus nanoparticles were added to bacteria growth at their lag phase and incubated for 2 hr. Nanoparticles induced membrane deformations and formed pores at the end of cell wall. (FIG. 6C-6E). It could be seen that with increased particle concentration, the cell was ruptured. As a comparator, bacteria were treated with 128 pM col/pho Janus nanoparticles. (FIG. 6F).

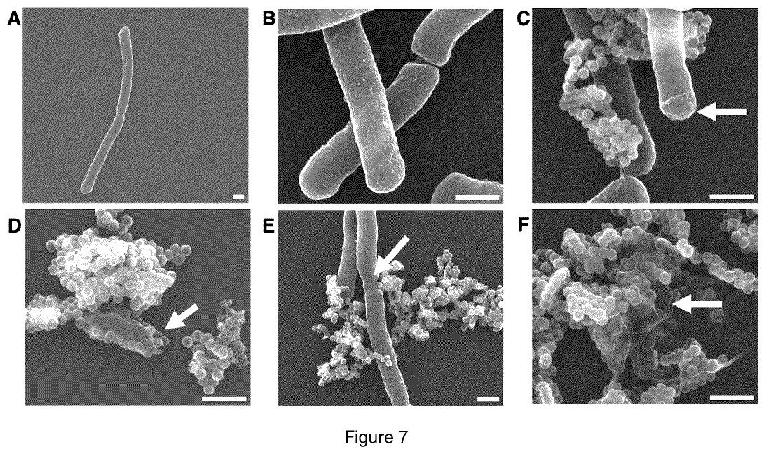

We also conducted the experiment and observed similar results with Gram-positive bacteria, Bacillus subtilis. Bacillus after interaction with 64 pM Janus nanoparticles forms pores at the end of the cell wall. (FIG. 7C-E) After bacteria are treated with 128 pM Janus nanoparticles, bacteria cell wall ruptured. (FIG. 7F)

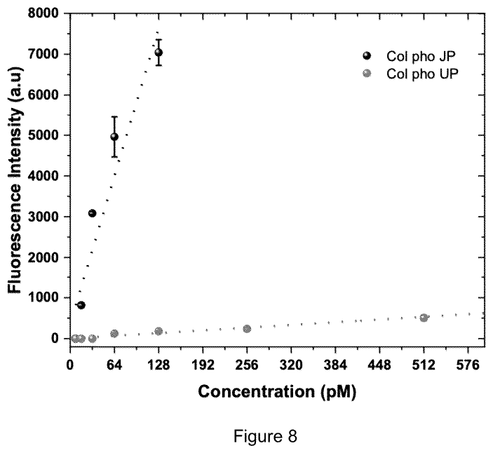

We studied Reactive Oxygen Species (ROS) in E. coli bacteria induced by colistin hydrophobic Janus nanoparticles. Bacteria were incubated with various concentrations of nanoparticles in the shaker for 2 hour and stained with Cell ROX dye for 30 minutes. Concentrations of uniform nanoparticles tested were 32 pM, 64 pM, 128 pM, and 512 pM as compared to 64 pM concentration of colistin hydrophobic Janus nanoparticles. An inverted Nikon Ti inverted microscope was used to image the cells. Janus nanoparticles induced significantly more ROS in E. coli bacteria than the uniform nanoparticles did. (FIG. 8)

### Materials and Methods Used in the Examples

Cells and reagents. E. coli (MG1655), S. aureus (Newman), B. subtilis (SB168) and V. cholerae (unpublished strain) were generously provided by the Gerdt lab at Indiana University-Bloomington. Luria-Bertani (LB) agar and Luria broth base were purchased from Invitrogen (Waltham, MA, USA). Amine-functionalized silica nanoparticles (100 nm) were procured from Nanocomposix (San Diego, CA, USA). Gold (99.99% purity) and chromium (99.99% purity) were purchased from Kurt J. Lesker, Co. (Jefferson Hills, PA, USA). Octadecanethiol, propidium iodide, octadecyltrimethoxysilane, colistin sulfate, G1 poly(amidoamine) dendrimer (20 wt % in methanol), and HEPES were purchased from Sigma-Aldrich (St. Louis, MO, USA). Ultrapure water (18 MΩ·cm) was used for all experiments.

Growth-based viability assay. Colonies of bacteria (E. coli MG1655, S. aureus Newman, V. cholerae (unpublished strain), B. subtilis SB168) were grown by streaking frozen glycerol:water stocks onto an LB agar plate and incubating the plate for 24 h at 37° C. Three colonies from the agar plate were then suspended in Luria broth to make stock suspensions, and the bacteria were grown to stationary phase overnight at 37° C. with constant shaking. Bacteria were then washed three times with 0.85% NaCl followed by three washes with 2 mM HEPES 25 mM NaCl (pH 7.2) immediately before each experiment. The growth-based viability assay, which determines bacteria viability based on the onset of logarithmic bacterial growth after exposure to a nanomaterial, was performed with modification as described by Qiu, et. al.²⁸ The exposure plate was made by making serial dilutions of nanoparticles suspended in 2 mM HEPES, 25 mM NaCl was performed on a 96-well plate. Bacteria were added to each well with particles for a final concentration of 1×10⁷ cfu/mL (4×10⁷ for B. subtilis). Two rows on each 96-well plate containing bacteria without nanoparticles were used to generate a calibration curve of the viability. In each row, 1×10⁷ cfu/mL (4×10⁷ for B. subtilis, treated as 100% viability) was added to the first well, and serial dilutions were performed in the subsequent wells. A row of wells with only media was added to each plate as a control for media sterility. The exposure plate was incubated for 2 h at 37° C. with 280 rpm shaking. After incubation, 20 uL of each well from the exposure plate was transferred into 180 uL each of growth media in a new 96-well plate (M9 minimal media for E. coli and V. cholerae or Luria broth for S. aureus and B. subtilis). Optical density at 600 nm, corresponding to number of bacteria, was collected for 16 h at 37° C. using a plate reader (Biotek Synergy H1). A code in R studio published by Qiu, et al. was used to analyze the growth curves to determine bacteria viability.²⁹

Propidium iodide assay. E. coli or B. subtilis, prepared as described above, were washed three times with 0.85% NaCl followed by three washes with 2 mM HEPES 25 mM NaCl (pH 7.2). Bacteria were then mixed with the desired volume of nanoparticles to reach a final incubation concentration of 1×10⁸ cfu/mL. Particles and bacteria were incubated for 2 h at 37° C. with shaking at 280 rpm. After incubation, propidium iodide was added to the bacteria-particle suspension (2 μM final concentration) and allowed to mix for 30 min. After staining, 10 μL of suspension was then sandwiched between a coverslip and microscope slide for fluorescence imaging. Fluorescence and differential interference contrast images were acquired using a Nikon Ti-E inverted microscope equipped with a 100×/1.49NA TIRF oil-immersion objective.

### REFERENCES

1. Antibiotic resistance threats in the United States, 2019.
2. Davies, J.; Davies, D., Origins and Evolution of Antibiotic Resistance. Microbiology and Molecular Biology Reviews 2010, 74 (3), 417-433.
3. Blair, J. M. A.; Webber, M. A.; Baylay, A. J.; Ogbolu, D. O.; Piddock, L. J. V., Molecular mechanisms of antibiotic resistance. Nature Reviews Microbiology 2015, 13 (1), 42-51.
4. Zhao, Y.; Tian, Y.; Cui, Y.; Liu, W.; Ma, W.; Jiang, X., Small Molecule-Capped Gold Nanoparticles as Potent Antibacterial Agents That Target Gram-Negative Bacteria. Journal of the American Chemical Society 2010, 132 (35), 12349-12356.
5. Hang, M. N.; Gunsolus, I. L.; Wayland, H.; Melby, E. S.; Mensch, A. C.; Hurley, K. R.; Pedersen, J. A.; Haynes, C. L.; Hamers, R. J., Impact of Nanoscale Lithium Nickel Manganese Cobalt Oxide (NMC) on the Bacterium Shewanella oneidensis MR-1. Chemistry of Materials 2016, 28 (4), 1092-1100.
6. Feng, Z. V.; Miller, B. R.; Linn, T. G.; Pho, T.; Hoang, K. N. L.; Hang, M. N.; Mitchell, S. L.; Hernandez, R. T.; Carlson, E. E.; Hamers, R. J., Biological impact of nanoscale lithium intercalating complex metal oxides to model bacterium B. subtilis. Environmental Science: Nano 2019, 6 (1), 305-314.
7. Arakha, M.; Pal, S.; Samantarrai, D.; Panigrahi, T. K.; Mallick, B. C.; Pramanik, K.; Mallick, B.; Jha, S., Antimicrobial activity of iron oxide nanoparticle upon modulation of nanoparticle-bacteria interface. Scientific Reports 2015, 5 (1), 14813.
8. Natan, M.; Gutman, O.; Lavi, R.; Margel, S.; Banin, E., Killing Mechanism of Stable N-Halamine Cross-Linked Polymethacrylamide Nanoparticles That Selectively Target Bacteria. ACS Nano 2015, 9 (2), 1175-1188.
9. Nederberg, F.; Zhang, Y.; Tan, J. P. K.; Xu, K.; Wang, H.; Yang, C.; Gao, S.; Guo, X. D.; Fukushima, K.; Li, L.; Hedrick, J. L.; Yang, Y.-Y., Biodegradable nanostructures with selective lysis of microbial membranes. Nature Chemistry 2011, 3 (5), 409-414.
10. Mei, L.; Lu, Z.; Zhang, X.; Li, C.; Jia, Y., Polymer-Ag Nanocomposites with Enhanced Antimicrobial Activity against Bacterial Infection. ACS Applied Materials & Interfaces 2014, 6 (18), 15813-15821.
11. Rai, A.; Pinto, S.; Velho, T. R.; Ferreira, A. F.; Moita, C.; Trivedi, U.; Evangelista, M.; Comune, M.; Rumbaugh, K. P.; Simbes, P. N.; Moita, L.; Ferreira, L., One-step synthesis of high-density peptide-conjugated gold nanoparticles with antimicrobial efficacy in a systemic infection model. Biomaterials 2016, 85, 99-110.
12. El Badawy, A. M.; Silva, R. G.; Morris, B.; Scheckel, K. G.; Suidan, M. T.; Tolaymat, T. M., Surface Charge-Dependent Toxicity of Silver Nanoparticles. Environmental Science & Technology 2011, 45 (1), 283-287.
13. Huo, S.; Jiang, Y.; Gupta, A.; Jiang, Z.; Landis, R. F.; Hou, S.; Liang, X.-J.; Rotello, V. M., Fully Zwitterionic Nanoparticle Antimicrobial Agents through Tuning of Core Size and Ligand Structure. ACS Nano 2016, 10 (9), 8732-8737.
14. Mensch, A. C.; Hernandez, R. T.; Kuether, J. E.; Torelli, M. D.; Feng, Z. V.; Hamers, R. J.; Pedersen, J. A., Natural Organic Matter Concentration Impacts the Interaction of Functionalized Diamond Nanoparticles with Model and Actual Bacterial Membranes. Environmental Science & Technology 2017, 51 (19), 11075-11084.
15. Goodman, C. M.; McCusker, C. D.; Yilmaz, T.; Rotello, V. M., Toxicity of Gold Nanoparticles Functionalized with Cationic and Anionic Side Chains. Bioconjugate Chemistry 2004, 15 (4), 897-900.
16. Gupta, A.; Landis, R. F.; Li, C.-H.; Schnurr, M.; Das, R.; Lee, Y.-W.; Yazdani, M.; Liu, Y.; Kozlova, A.; Rotello, V. M., Engineered Polymer Nanoparticles with Unprecedented Antimicrobial Efficacy and Therapeutic Indices against Multidrug-Resistant Bacteria and Biofilms. Journal of the American Chemical Society 2018, 140 (38), 12137-12143.
17. Hayden, S. C.; Zhao, G.; Saha, K.; Phillips, R. L.; Li, X.; Miranda, O. R.; Rotello, V. M.; El-Sayed, M. A.; Schmidt-Krey, I.; Bunz, U. H. F., Aggregation and Interaction of Cationic Nanoparticles on Bacterial Surfaces. Journal of the American Chemical Society 2012, 134 (16), 6920-6923.
18. Li, X.; Robinson, S. M.; Gupta, A.; Saha, K.; Jiang, Z.; Moyano, D. F.; Sahar, A.; Riley, M. A.; Rotello, V. M., Functional Gold Nanoparticles as Potent Antimicrobial Agents against Multi-Drug-Resistant Bacteria. ACS Nano 2014, 8 (10), 10682-10686.
19. Pillai, P. P.; Kowalczyk, B.; Kandere-Grzybowska, K.; Borkowska, M.; Grzybowski, B. A., Engineering Gram Selectivity of Mixed-Charge Gold Nanoparticles by Tuning the Balance of Surface Charges. Angewandte Chemie International Edition 2016, 55 (30), 8610-8614.
20. Zhang, X.; Fu, Q.; Duan, H.; Song, J.; Yang, H., Janus Nanoparticles: From Fabrication to (Bio)Applications. ACS Nano 2021, 15 (4), 6147-6191.
21. Lee, K.; Zhang, L.; Yi, Y.; Wang, X.; Yu, Y., Rupture of Lipid Membranes Induced by Amphiphilic Janus Nanoparticles. ACS Nano 2018, 12 (4), 3646-3657.
22. Wiemann, J. T.; Shen, Z.; Ye, H.; Li, Y.; Yu, Y., Membrane poration, wrinkling, and compression: deformations of lipid vesicles induced by amphiphilic Janus nanoparticles. Nanoscale 2020, 12 (39), 20326-20336.
23. Lee, K.; Yu, Y., Lipid Bilayer Disruption by Amphiphilic Janus Nanoparticles: The Role of Janus Balance. Langmuir 2018, 34 (41), 12387-12393.
24. Sabnis, A.; Hagart, K. L. H.; Klockner, A.; Becce, M.; Evans, L. E.; Furniss, R. C. D.; Mavridou, D. A. I.; Murphy, R.; Stevens, M. M.; Davies, J. C.; Larrouy-Maumus, G. J.; Clarke, T. B.; Edwards, A. M., Colistin kills bacteria by targeting lipopolysaccharide in the cytoplasmic membrane. eLife 2021, 10, e65836.
25. El-Sayed Ahmed, M. A. E.-G.; Zhong, L.-L.; Shen, C.; Yang, Y.; Doi, Y.; Tian, G.-B., Colistin and its role in the Era of antibiotic resistance: an extended review (2000-2019). Emerging Microbes & Infections 2020, 9 (1), 868-885.
26. Poli, E.; Jong, K. H.; Hassanali, A., Charge transfer as a ubiquitous mechanism in determining the negative charge at hydrophobic interfaces. Nature Communications 2020, 11 (1), 901.
27. Bazant, M. Z., Induced-charge electrokinetic phenomena. Courses Lect.—Int. Cent. Mech. Sci. 2011, 530 (Electrokinetics and Electrohydrodynamics in Microsystems), 221-297.
28. Qiu, T. A.; Nguyen, T. H. T.; Hudson-Smith, N. V.; Clement, P. L.; Forester, D.-C.; Frew, H.; Hang, M. N.; Murphy, C. J.; Hamers, R. J.; Feng, Z. V.; Haynes, C. L., Growth-Based Bacterial Viability Assay for Interference-Free and High-Throughput Toxicity Screening of Nanomaterials. Anal. Chem. 2017, 89 (3), 2057-2064.
29. Lopez, A. I.; Reins, R. Y.; McDermott, A. M.; Trautner, B. W.; Cai, C., Antibacterial activity and cytotoxicity of PEGylated poly(amidoamine) dendrimers. Molecular BioSystems 2009, 5 (10), 1148-1156.

### EQUIVALENTS AND SCOPE

Those skilled in the art will recognize or be able to ascertain using no more than routine experimentation many equivalents to the specific embodiments described herein. The scope of the present invention is not intended to be limited to the above, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should be understood that, in general, where the invention, or aspects of the invention is/are referred to as comprising particular elements, features, etc., certain embodiments of the invention or aspects of the invention consist, or consist essentially of, such elements, features, etc. For purposes of simplicity, those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranged can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed. Unless states otherwise of clearly implied otherwise the term ‘about’ means plus or minus 10 percent, for example about 1.0 encompasses the ranges of values from 0.9 to 1.1.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the method of the invention can be excluded from any one or more claims, for any reason, whether or not related to the existence of prior art.

Each of the foregoing patents, patent applications and references is hereby incorporated by reference, particularly for the teaching referenced herein.

I claim:

1. A compound, comprising:
a nanoparticle;
a charged moiety; and
a hydrophobic moiety, wherein the charged moiety and the hydrophobic moiety are attached to the nanoparticle independently of one another.

2. The compound according to claim 1, wherein the charged moiety is attached to a first hemisphere and the hydrophobic moiety is attached to the opposite hemisphere of the nanoparticle.

3. The compounds according to claims 1-2, wherein hydrophobic moiety comprises at least one hydrophobic alkyl chain.

4. The compound according to claim 3, wherein the at least one hydrophobic alky chain is selected from the group consisting of: linear chains, branched chains, or polymers.

5. The compounds according to claims 3-4, wherein one of the hydrophobic alkyl moiety, includes 4 or more carbon-carbon bonds.

6. The compound according to claim 1-2, wherein the charged moiety is an antibiotic.

7. The compound according to claim 6, wherein the antibiotic is selected from the group consisting of: polymyxin antibiotics, antimicrobial peptides, and steroid antibiotic, wherein the antibiotic exhibit a positive charge.

8. The compound according to claim 7, wherein the antibiotic is colistin.

9. The compounds according to claims 1-2, wherein the charged moiety is a cationic polymer.

10. The compound according to claim 11, wherein the cationic polymer is at least one cationic polymer selected from the group consisting of: cationic poly(amidoamine), cationic linear polymers, cationic branched polymers, cationic dendrimers, cationic polypeptide, or other cationic nanoparticles.

11. The compounds according to claims 1-2, wherein the compounds kill Gram-negative bacteria.

12. The compounds according to claims 1-2, wherein the compounds kill Gram-positive bacteria.

13. A method of killing bacteria, comprising the steps of:
providing at least one compound according to claims 1-2; and
contacting the least one compound with a bacteria cell.

14. The method according to claim 13, wherein the bacteria are selected from the group consisting of: Gram-negative and Gram-positive bacteria.

15. A method of treating a patient comprising the steps of:
providing at least one compound according to claims 1-2; and
administering at least one therapeutically effective dose of the least one compound to a patent, where the patient is diagnosed with a bacterial infection.

16. The method according to claim 15, wherein the therapeutically effective dose is within the picomolar to micron-molar range.

17. The methods according to claims 15-16, wherein the bacterial infection is caused by at least one bacterium selected from the group consisting of: Gram-negative and Gram-positive bacteria.

18. The methods according to claims 15-17, wherein the patient is a human or an animal.

19. The methods according to claims 15-18, where in the therapeutically effect dose is administered topically.

21. The methods according to claims 15-18, where in the therapeutically effect dose is administered orally.

22. The methods according to claims 15-18, where in the therapeutically effect dose is administered intravenously.
